(19) United States
(12) Patent Application Publication
(10) Pub. No.: US 2003/0177089 A1
(43) Pub. Date: Sep. 18, 2003

(54) NON-SURGICAL METHOD FOR BREAST AUGMENTATION

(76) Inventor: MARIANTHI GIAKOUMAKIS, MONTREAL (CA)

Correspondence Address: DOUGLAS R HANSCOM, JONES TULLAR & COOPER, P O BOX 2266 EADS STATION, ARLINGTON, VA, 22202

(21) Appl. No.: 08/876,437

(22) Filed: Jun. 16, 1997

Related U.S. Application Data
(63) Continuation of application No. 08/731,008, filed on Oct. 9, 1996, now abandoned.

(51) Int. Cl. G06F 17/60; A61B 19/00

(52) U.S. Cl. 705/40

(57) ABSTRACT

Breast augmentation usually involves a surgical procedure. A non-surgical method for breast augmentation involves the use of cocoa butter and Vitamin E, either subsequently to each other or in combination.

### FIELD OF THE INVENTION

The present invention relates to a method for breast augmentation, and in particular to a non-surgical method for breast augmentation.

### BACKGROUND OF THE INVENTION

Breast augmentation is a growing industry. In the United States alone, it is estimated that each year over one million women undergo surgery for breast enlargement.

The most commonly used procedure for breast augmentation is mammaplastry, which is, surgical augmentation of the breasts. This procedure generally involves making a surgical incision to create a pocket in the breast followed by insertion of a mammary prosthesis.

Present day prosthesis are usually made from a polysilicone elastomer sac filled with a silicone gel or mixture thereof. However, such implants are not entirely safe. They have been know to leak and rupture, causing long-term adverse effects on the body.

Saline-filled implants are also popular. They are considered safer because saline solution is similar to other body fluids and leakage thereof will simply be absorbed without harm. However, saline-filled implants do not feel or look as natural as gel implants. They tend to wrinkle or ripple under the skin preventing the breast from moving and hanging naturally. Moreover, it is believed that saline implants have a higher rate of leaking and deflation than silicone gel implants. Thus frequent surgery may be required to replace the implants.

Because of all the problems associated with implants, alternatives have been sought. The latest in implant alternatives is a new technique known by the acronym BAMBI (bilateral augmentation mammoplastry by injection). The BAMBI method involves surgery and entails liposuctioning fat from the body, for example from the buttocks or thighs for injection into the chest. Although some plastic surgeons believe the method is safe, others do not. The surgery may cause dangerous infection, and the fat can calcify resulting in masses of scar tissue which can mimic or mask breast cancer. This, in turn, can lead to unnecessary biopsies if there is no cancer, or delay detection if there is cancer.

More natural non-intrusive methods have also been sought. For example, it has been found that cocoa butter augments breast size. The use of cocoa butter for breast augmentation is mentioned on page 285 of the Edgar Cayce Handbook for Health Through Drugless Therapy. Cayce states, in his reading numbered 934-13, that a method to enlarge the breast is obtained, as follows: “This is a little bit late in beginning [the woman was aged thirty-two] but if there is the massage of the mammary glands with cocoa butter—not on the breast itself, but under the arm and lower and in the area between the breast—you can get'em as big or as little as you wish.” However, it had been found by the applicant that augmentation of the breast using Cayce's method alone did not last, i.e. the breast does not remain enlarged with the application of cocoa butter for long and eventually returns to the same size, as prior to the application of cocoa butter, in the following few days.

### SUMMARY OF THE INVENTION

An object of the present invention is to provide a non-surgical method for breast size augmentation and to sustain the augmented breast size.

In accordance with one aspect of the present invention there is provided a method for augmenting the size of a human breast and sustaining the augmented breast size comprising the steps of applying cocoa butter to the breast for augmenting the size of the breast, and applying Vitamin E to the breast for sustaining the augmented breast size.

In accordance with another aspect of the present invention there is provided a composition for augmenting the size of a human breast and sustaining the augmented breast size comprising cocoa butter and Vitamin E.

In accordance with another aspect of the invention there is provided the use of cocoa butter and Vitamin E for augmenting the breast size of a human and sustaining the augmented breast size.

Preferably, the cocoa butter is applied in an amount sufficient to achieve the desired augmentation of the breast and the Vitamin E is applied in an amount sufficient to sustain the desired augmented breast size.

In one embodiment of the invention, the cocoa butter and Vitamin E are simultaneously applied to the breast.

In another embodiment of the invention the cocoa butter is first applied to the breast, and the Vitamin E is applied subsequently.

Preferably, the proportion of cocoa butter to Vitamin E is 18:1.

The advantages of the present invention are that there are no surgical procedures necessary, hence eliminating all the potential problems, either physical, emotional or psychological, that might come with having surgery. Therefore, the present invention provides a safer and more natural method than surgery for enlarging the size of the human breast and sustaining the augmented breast size. Moreover, the method is simple and inexpensive. In addition, there is the benefit derived from the psychological satisfaction and emotional well being women would experience from augmenting their breast size by using this healthier and more natural invention than surgical breast augmentation. Therefore, this method is an advance to man kind because it is a more natural and safer method of breast augmentation than breast augmentation attained by surgery.

### DETAILED DESCRIPTION OF THE INVENTION

The preferred method of the present invention includes applying cocoa butter (a fatty substance obtained from cacao seeds), for example Derma-E*, and Vitamin E, for example d-alpha-tocopherol acetate by Jamieson Natural Source*, to the breast for enlargement thereof. The cocoa butter and Vitamin E can be applied separately or as a mixture. The breast includes an area generally between the second and seventh rib and extending between the axilla (armpit) and the sternum. Further to Mr. Cayce's method, it has been found by the applicant that the application of the cocoa butter also on the globe of the breast itself promotes enlargement thereof. It is the applicant's belief that the cocoa butter is absorbed into the breast, hence enlarging the size of the breast by the breast assimilating the cocoa butter and storing the cocoa butter around the gland lobules of the mammary gland connected to the breast therein, by means of the breast assimilating the cocoa butter as fat. Therefore, further to Mr. Cayce's method, it is desirable to apply cocoa butter also to the globe of the breast in order to augment the size of the breast. Preferably, the application of cocoa butter includes gentle rubbing on the skin of the breast, that is the area of application, to facilitate the absorption of the cocoa butter into the breast to augment the breast in size. Furthermore, in the application of cocoa butter or Vitamin E it is preferable to apply cocoa butter or vitamin E in thin layers at a time to create a friction to the skin that lessens the amount of time needed for the absorption of cocoa butter or vitamin E into the breast. Also, first heating the breast area before the application of cocoa butter or vitamin E lessens the amount of time needed for the absorption of either ingredient into the breast. *Trade-marks

The amount of cocoa butter used is directly proportional to the augmentation of the breast's size. It has been found that approximately 50 g of cocoa butter per breast enlarges each breast from an A size bra cup to a B size bra cup. For such an amount, the application time for the applicant, at a younger age of approximately 30 years old and under, was approximately 11 consecutive hours of application of the cocoa butter. This was before the applicant's discovery that Vitamin E would sustain the augmented breast size, and so the breast would reduce to the same breast size as before the application of the cocoa butter. However, at an older age, approximately the age of thirty and over, the applicant had found that response time of the body to the application of cocoa butter took significantly longer for the breast to augment in size. At the age of 35 years, it would then take the applicant 4 to 6 hours to absorb about 7 g of cocoa butter.

Following the application of the cocoa butter, Vitamin E is applied to the breast. Further to Cayce's statement, it has been found that the use of Vitamin E sustains the augmented breast size. Vitamin E sustains the size of the breast augmented by the application of cocoa butter to the breast. It had been found by the applicant that the omission of the use of Vitamin E caused the body to lose all the augmentation in breast size derived from the application of the cocoa butter in the days that followed the application of the cocoa butter.

It is the applicant's experience at the age of 35 that the application of approximately 400 mg of Vitamin E was satisfactory to sustain the breast size augmented by the application to the breast and absorption by the breast, of approximately 7 g of cocoa butter. Therefore giving a proportion of cocoa butter to Vitamin E of about 18:1. It has been found by the applicant that subsequent applications of cocoa butter and Vitamin E to the breast further augmented and sustained the breast in size and that at no time did the augmented breast size reduce again in size from one application sitting to the next. It has been found that simply smearing the Vitamin E to the breast area without the use of gentle rubbing to aid in the absorption of the Vitamin E is also sufficient to sustain the size of the breast augmented by the application of cocoa butter.

Also, the application of Vitamin E does not necessarily need to be applied just after the application of cocoa butter for this method to work. Even the application of Vitamin E to the breast prior to the application of the cocoa butter has also been found by the applicant to have the desired effect in sustaining the breast size augmented by the application of cocoa butter.

Augmentation of the size of the breast and sustaining the augmented size can also be achieved using a composition of cocoa butter and Vitamin E in an approximate proportion of 18 to 1. The composition is produced by melting the cocoa butter at very low heat, approximately 40° Fahrenheit, and the Vitamin E is added thereto to first obtain a mixture. The mixture was stirred for 30 to 40 seconds and let stand to cool before use to obtain the final composition.

It will be recognized by one skilled in the art that the above mentioned amounts, quantity and proportion of ingredients of cocoa butter and Vitamin E are merely guidelines and are not limiting to the scope of the present patent application. The exact amounts and proportions of cocoa butter and Vitamin E necessary to augment the breast size and sustain the augmented breast size can vary from one person to another, depending on, for example, different body types and different body compositions, and the person's age.

While a preferred embodiment of the invention has been described, it is understood that all alternatives and modifications may be made thereto and fall within the scope of the invention.

I claim:

1. A method for augmenting the size of a human breast and sustaining the augmented breast size comprising the steps of:
applying cocoa butter to the breast for augmenting the size of the breast; and
applying Vitamin E to the breast for sustaining the augmented breast size.

2. A method of claim 1, wherein the cocoa butter is applied in an amount sufficient to achieve the desired augmentation of the breast and the Vitamin E is applied in an amount sufficient to sustain the desired augmented breast size.

3. The method of claim 1, wherein the cocoa butter and Vitamin E are simultaneously applied to the breast.

4. The method of claim 1, wherein the cocoa butter is first applied to the breast, and the Vitamin E is applied subsequently.

5. The method of claim 1, wherein the proportion of cocoa butter to Vitamin E is 18:1.

6. The method of claim 1, wherein 7 g of cocoa butter and 400 mg of Vitamin E are applied to the breast.

7. A composition for augmenting the size of a human breast and sustaining the augmented breast size comprising cocoa butter and Vitamin E.

8. The composition in claim 7, wherein the proportion of cocoa butter to Vitamin E is 18:1.

9. The use of cocoa butter and Vitamin E for augmenting the breast size of a human and sustaining the augmented breast size.
